(19) United States
(12) Patent Application Publication
(10) Pub. No.: US 2024/0170104 A1
(43) Pub. Date: May 23, 2024

(54) METHOD AND SYSTEM FOR PREDICTING ADVERSE DRUG-DRUG INTERACTIONS BY RECOVERING THE MULTI-ATTRIBUTE INFORMATION OF DRUGS, AND MEDIUM

(71) Applicant: University of Electronic Science and Technology of China, Chengdu (CN)

(72) Inventors: Jiajing ZHU, Chengdu (CN); Yongguo LIU, Chengdu (CN); Yun ZHANG, Chengdu (CN); Qiaoqin LI, Chengdu (CN); Zhi CHEN, Chengdu (CN)

(21) Appl. No.: 18/325,572

(22) Filed: May 30, 2023

(30) Foreign Application Priority Data
Nov. 16, 2022 (CN) 202211434048.4

(51) Int. Cl. G16C 20/30; G16C 20/70

(52) U.S. Cl. CPC G16C 20/30; G16C 20/70

(57) ABSTRACT

The present invention discloses the method and system for predicting adverse drug-drug interactions by recovering the multi-attribute information of drugs and the medium. The method includes: collecting adverse drug-drug interactions data and multi-attribute data of drugs; constructing the recovery model of multi-attribute absent feature of drugs; correcting the recovery model of multi-attribute absent feature of drugs by the cosine similarity regularization term, and solving the corrected recovery model to obtain the common features and unique features of multi-attribute information of drugs; obtaining the multi-attribute information of two drugs, calculating common features and unique features of their multi-attribute information as the inputs of the prediction model to predict their adverse drug-drug interactions. The present invention improves the accuracy of the prediction of the adverse drug-drug interactions, promotes the experimental study of the adverse drug-drug interactions, and ensures the safety of medication.

### CROSS REFERENCE TO RELATED APPLICATION

This application claims the benefit of Chinese Patent Application No. 202211434048.4, filed on Nov. 16, 2022, the disclosure of which is incorporated by reference herein in its entirety.

### TECHNICAL FIELD

The present invention relates to an adverse interaction prediction technology, in particular to a method and system for predicting adverse drug-drug interactions by recovering the multi-attribute information of drugs, and a medium.

### BACKGROUND

Adverse drug-drug interactions mean that the efficacy or pharmacology of one drug is destroyed by the other drug during concomitant medication, so as to change the drug's original systemic processes, tissues or organs' perception of the drug and the chemical properties of the drug, resulting in adverse interactions or side effects harmful to human body.

At present, adverse drug-drug interactions have become an important factor in delaying disease treatment, aggravating patients' conditions, and affecting patients' morbidity and mortality. The study of adverse drug-drug interactions has gradually attracted the attention of relevant medical and health institutions, and has become the focus of current medical and health research. Pharmaceutical enterprises have invested a lot of money to carry out clinical experiments of adverse drug-drug interactions in the drug research and development stage to solve this problem. At present, there are mainly two kinds of research methods for predicting adverse drug-drug interactions, namely knowledge-based method and similarity-based method.

Knowledge-based method is usually based on data mining and natural language processing technologies to identify adverse drug-drug interactions through biomedical texts, electronic medical cases, biological heterogeneous databases and FDA adverse event reporting system. This method relies on the data accumulation of adverse drug-drug interactions in clinical practice, and is intended to identify adverse drug-drug interactions from massive unformatted data. While in the similarity-based method, attribute information of drugs is first extracted from a drug database, and attribute similarity scores are calculated based on the relationship between attribute information of drugs, then a machine learning model is designed to explore the potential relationship between the attribute similarity scores and adverse drug-drug interactions to predict potential adverse drug-drug interactions. This method can predict adverse drug-drug interactions only depending on attribute information of drugs, without needing a large amount of previous adverse interaction data.

However, in the prior art, the drugs used in the process of building the prediction model of adverse drug-drug interactions based on drug attribute features usually have complete attribute feature information. Drugs with absent attribute features have not been considered. Different attribute information of drugs usually comes from different heterogeneous databases. The number of drugs and the attribute information recorded in different databases are significantly different. For example, the number of drugs in the database SIDER is far less than that in the database DrugBank. It can be seen that the majority of drugs with molecular structure, target and enzyme information in DrugBank lack side effect information, resulting in a large number of drug side effect information missing. In addition, other attributes of drugs are also absent due to differences in the number and type of drugs between different databases. The number of drugs with complete attribute feature information will gradually decrease, and the lack of drug attribute feature information will become more and more serious with the increasing attribute factors considered in the model.

Therefore, if we continue to predict adverse drug-drug interactions with this method, it will reduce the efficiency of adverse interaction research, and lead to inaccurate prediction results, and even lead to drug safety accidents in some serious conditions.

### SUMMARY

The technical problem to be solved by the present invention that is drugs with absent attribute features have not been considered in the prediction of adverse drug-drug interactions in the existing technology with the knowledge-based method or similarity-based method, and the absent attribute features of different drugs are often different, therefore, if we continue to use the existing technology for prediction, it will reduce the efficiency of adverse interaction research, and lead to inaccurate prediction results, and even lead to drug safety accidents. The present invention aims to provide a method and system for predicting adverse drug-drug interactions by recovering the multi-attribute information of drugs, and a medium, so as to improve the efficiency of the research on adverse drug-drug interactions, improve the accuracy of the prediction of the adverse drug-drug interactions, and ensure the safety of medication.

The present invention is implemented by using following technical solutions:

A method for predicting adverse drug-drug interactions by recovering the multi-attribute information of drugs, where the method comprises: collecting adverse drug-drug interactions data and multi-attribute data of drugs; constructing a recovery model of multi-attribute absent feature of drugs based on common features and unique features of multi-attribute information of drugs; correcting the recovery model of multi-attribute absent feature of drugs by a cosine similarity regularization term, and solving the corrected recovery model by Lagrange function, alternating direction method of multipliers and nonnegative matrix factorization to obtain the common features and unique features of multi-attribute information of drugs; constructing a prediction model based on the common features and unique features of multi-attribute information of drugs and the adverse drug-drug interactions data; and obtaining the multi-attribute information of two drugs, calculating common features and unique features of their multi-attribute information as the inputs of the prediction model to predict their adverse drug-drug interactions.

In the traditional prediction solution of adverse drug-drug interactions, knowledge-based method or similarity-based method are usually used. However, the drugs with absent attribute features have not been considered when predicting adverse drug-drug interactions with these methods, and the absent attribute features of different drugs are often vastly different. Therefore, if we continue to predict adverse drug-drug interactions using drugs with absent attribute features, not only will we not be able to deeply analyze the potential relationship between multi-attribute-information of drugs and adverse interactions, but will also lead to inaccurate prediction results, and even lead to drug safety accidents. The present invention provides an adverse interactions prediction method by recovering the multi-attribute information of drugs, which recovers the absent features of drugs by constructing a recovery model of multi-attribute absent feature of drugs, and then predicts adverse interactions among drugs with the recovered attribute features. This will not only improve the accuracy of the prediction of the adverse drug-drug interactions, but also promote the experimental study of the adverse drug-drug interactions, and ensure the safety of medication.

Preferably, the specific step of constructing the recovery model of multi-attribute absent feature of drugs comprises: employing multi-attribute information of drugs and constructing a basic model based on the relationship between the common features and unique features of an attribute and its original feature space; and using KL divergence to measure a distribution difference between unique features of different attributes, and processing the basic model by the distribution difference to obtain the recovery model of multi-attribute absent feature of drugs based on common features and unique features.

Preferably, a specific method for obtaining the common features and unique features of the multi-attribute information of drugs comprises: correcting the recovery model of multi-attribute absent feature of drugs by a cosine similarity regularization term to obtain a corrected model; solving the corrected model by Lagrange function, alternating direction method of multipliers and nonnegative matrix factorization to obtain the update solutions of recovered feature spaces of different attributes and common features and unique features of multi-attribute information, as well as the reconstruction coefficient matrices of the feature spaces of different attributes; and iteratively updating variables of the corrected model until it reaches the maximum number of iteration or the difference of the objective function of the model less than a threshold to obtain the common features and unique features of the multi-attribute information of drugs.

Preferably, the multi-attribute information of drugs comprises molecular structure, target, pathway, side effect, phenotype and disease data.

Preferably, a specific expression of the recovery model of multi-attribute absent feature of drugs based on common features and unique features is:

$${min}_{{X}^{m} , P , {Q}^{m} , {U}^{m}} {\sum }_{m = 1}^{M} [ {‖ {X}^{m} - ( P + {Q}^{m} ) {U}^{m} ‖}_{F}^{2} + {α}^{m} {‖ {U}^{m} ‖}_{0} ] - β {\sum }_{n , m}^{M} [ KL ( {Q}^{m} ‖ {Q}^{n} ) + KL ( {Q}^{n} ‖ {Q}^{m} ) ]$$

$$s . t . {H}_{E}^{m} {X}^{m} = {X}_{E}^{m} , P \geq 0 , {Q}^{m} \geq 0 , m = 1 , … , M$$

∥·∥_{F}² represents the Frobenius norm of the matrix, ∥·∥₀ represents the l₀ norm of the matrix, P represents the common features of multi-attribute information of drugs, Q^{m} represents the unique features of the m-th attribute of drugs, U^{m} represents a reconstruction coefficient matrix of original feature space X^{m} based on the common features and unique features in the m-th attribute, X^{m} represents original feature space of the m-th attribute of drugs, X_{E}^{m} represents the known feature information of the m-th attribute of drugs, KL represents divergence,

$${H}_{E}^{m} ∈ {\{ 0 , 1 \}}^{{N}_{E}^{m} \times N}$$

represents a marker matrix of X_{E}^{m}, H_{E}^{m}X^{m=X}_{E}^{m} represents that drugs with known features are extracted from X^{m} and sorted by index to obtain X_{E}^{m}. α^{m} represents a sparse regularization parameter of the reconstruction coefficient matrix of the m-th attribute, β represents a regularization parameter of KL divergence between unique features of different attributes.

Preferably, a specific expression of the corrected model is:

$${min}_{{X}^{m} , P , {Q}^{m} , {U}^{m}} {\sum }_{m = 1}^{M} [ {‖ {X}^{m} - ( P + {Q}^{m} ) {U}^{m} ‖}_{F}^{2} + {α}^{m} {‖ {U}^{m} ‖}_{0} + {γ}^{m} {\sum }_{i , j}^{N} {‖ {( P + {Q}^{m} )}_{i \cdot } - {( P + {Q}^{m} )}_{j \cdot } ‖}_{2}^{2} {S}^{m} ( {d}_{i} , {d}_{j} ) ] - β {\sum }_{n , m}^{M} [ KL ( {Q}^{m} ‖ {Q}^{n} ) + KL ( {Q}^{n} ‖ {Q}^{m} ) ]$$

$$s . t . {H}_{E}^{m} {X}^{m} = {X}_{E}^{m} , P \geq 0 , {Q}^{m} \geq 0 , {U}^{m} \geq 0 , m = 1 , … , M$$

S^{m}(dᵢ,dⱼ) can be regarded as standardized cosine similarity between vectors Xᵢ^{m.} and X_{j.}^{m}, (P+Q^{m})_{i.} and (P+Q^{m})_{j.} are a combined representation of common features and unique features of drugs dᵢ and dⱼ, respectively, γ^{m} represents a regularization parameter of cosine similarity regularization term of the m-th attribute, and ∥·∥₂² represents the l₂ norm of a vector.

Preferably, a specific expression of the prediction model is:

$${\sum }_{{\vert {r}^{ij} ‖}_{0} \neq 0} {‖ {r}^{ij} - ( {\overline{r}}^{ij} + {\tilde{r}}^{ij} ) ‖}_{2}^{2}$$

[symbol]

represents contribution of common features to adverse drug-drug interactions, [symbol] represents contribution of unique features to adverse drug-drug interactions, and r^{ij} represents a relationship of adverse drug-drug interactions.

Preferably, a specific expression of [symbol] is:

[symbol]

=λ×E̅×₁P_{i.}×₂P_{j.}

a specific expression of [symbol] is:

$${\tilde{r}}^{ij} = {\sum }_{m = 1}^{M} {w}_{m} \times {E}_{m} {\times }_{1} {Q}_{i \cdot }^{m} {\times }_{2} {Q}_{j \cdot }^{m}$$

P_{i.} represents common features of multi-attribute of drug dᵢ, P_{j.} represents common features of multi-attribute of drug dⱼ, Q_{i.}^{m} represents unique features of the m-th attribute of dᵢ, Q_{j.}^{m} represents unique features of the m-th attribute of drug dⱼ, wₘ represents contribution of unique features Q_{i.}^{m} and Q_{j.}^{m} of the m-th attribute to adverse interactions between dᵢ and dⱼ, λ represents contribution of common features P_{i.} and P_{j.} to the adverse interactions between dᵢ and dⱼ, Ē represents tensor of the common features—adverse interactions that indicates a potential relationship between the common features and adverse interactions, Eₘ represents a potential relationship between the unique features of the m-th attribute and adverse interactions, and xₖ represents the product of the k-th order of the tensor and the vector, where k∈{1,2}.

The present invention also provides a system for predicting adverse drug-drug interactions by recovering the multi-attribute information of drugs, comprising a data collecting module, a recovery model construction module, an analysis module, a prediction model construction module and a prediction module; wherein, the data collecting module is configured to collecting adverse drug-drug interactions data and multi-attribute data of drugs; the recovery model construction module is configured to construct a recovery model of multi-attribute absent feature of drugs based on common features and unique features of multi-attribute data of drugs; the analysis module is configured to correct the recovery model by a cosine similarity regularization term, and solve the corrected recovery model by Lagrange function, alternating direction method of multipliers and nonnegative matrix factorization to obtain the common features and unique features of the multi-attribute information of drugs; the prediction model construction module is configured to construct a prediction model based on the common features and unique features of multi-attribute information of drugs and the adverse drug-drug interactions data; and the prediction module is configured to obtain the multi-attribute information of two drugs, calculate common features and unique features of their multi-attribute information as the inputs of the prediction model to predict their adverse drug-drug interactions.

The present invention also provides a computer storage medium storing a computing program, where the method described above is implemented when the computer program is executed by a processor.

The present invention has the following advantages compared with the existing technology:

The present invention provides a prediction method and system for adverse drug-drug interactions by recovering the multi-attribute information of drugs, and a medium, which recovers the absent features of drugs by constructing a recovery model of multi-attribute absent feature of drugs, and then predicts adverse interactions among drugs with the recovered attribute features. This will not only improve the accuracy of the prediction of the adverse drug-drug interactions, but also promote the experimental study of the adverse drug-drug interactions, and ensure the safety of medication.

### BRIEF DESCRIPTION OF DRAWINGS

The following will briefly introduce the drawings needed in the embodiments to more clearly illustrate the technical solutions of the embodiments of the present invention. It should be understood that the following figures illustrate only some embodiments of the present invention, and therefore should not be considered as limiting the scope. A person of ordinary skill in the art may still derive other related drawings from these accompanying drawings without creative efforts.

FIG. 1 is a schematic diagram of a prediction method.

FIG. 2 is a frame diagram of the recovery model of multi-attribute absent feature of drugs.

FIG. 3 is a frame diagram of a prediction model for adverse drug-drug interactions based on common features and unique features.

FIG. 4 shows some predicted adverse drug-drug interactions.

FIG. 5 shows some predicted adverse drug-drug interactions.

### DETAILED DESCRIPTION

The present invention is further described in combination with embodiments and figures to make the purpose, technical solution and advantages of the present invention more clear. The schematic embodiments of the present invention and their descriptions are only used to explain the present invention, and are not used to limit the present invention.

### Embodiment 1

In the traditional prediction solution of adverse drug-drug interactions, knowledge-based method or similarity-based method are usually used. However, the drugs with absent attribute features have not been considered when predicting adverse drug-drug interactions with these methods, and the absent attribute features of different drugs are often different. Therefore, if we continue to predict adverse drug-drug interactions using drugs with absent attribute features, not only will we not be able to deeply analyze the potential relationship between multi-attribute information of drugs and adverse interactions, but will also lead to inaccurate prediction results, and even lead to drug safety accidents.

This embodiment provides a prediction method for adverse drug-drug interactions by recovering the multi-attribute information of drugs, which effectively recovers the absent features based on multi-attribute information of drugs, and establishes a recovery model of multi-attribute absent feature of drugs based on common features and unique features. Based on the common features and unique features of attributes, an adverse interaction prediction model based on multi-attribute information of drugs is established to explore contribution of different attributes to adverse interactions and predict adverse drug-drug interactions. This method can provide data support for the experimental study of adverse drug-drug interactions, improve the clinical experimental study of adverse drug-drug interactions, and is of great significance for reducing the incidence of adverse drug-drug interactions, improving the efficiency of adverse drug-drug interactions research and improving safety of medication.

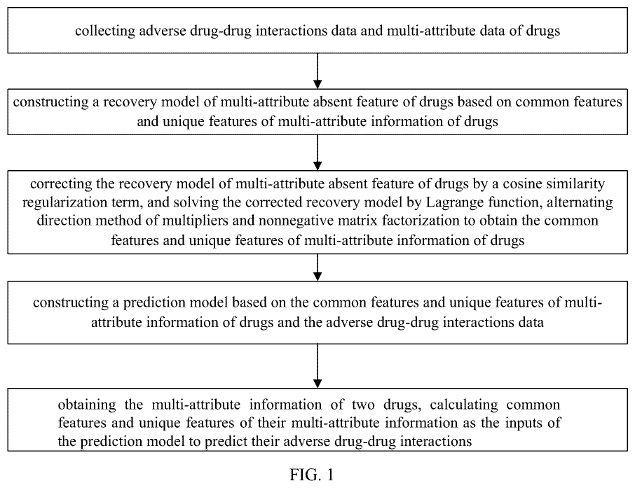

The prediction method is specifically shown in FIG. 1, and includes steps of:
S1: collecting adverse drug-drug interactions data and multi-attribute data of drugs.

The multi-attribute data includes molecular structure, target, pathway, side effect, phenotype and disease data.

Data of adverse drug-drug interactions is collected in the TWOSIDES database in step S1. Adverse interactions caused by the combination of two drugs are recorded in the TWOSIDES database. The molecular structure and target information of drugs come from the DrugBank database, the pathway and disease information of drugs come from the KEGG database, the side effect information of drugs comes from the SIDER database, and the phenotype information of drugs comes from the CTD database. PubChem substructure fingerprint is used to encode the SMILES molecular formula of drugs for the molecular structure information of drugs. Each drug contains 881 dimensional substructure information. Other attribute information of drugs is represented by a binary vector, and vector elements 1 and 0 respectively indicate whether a drug contains feature information of a corresponding attribute. The source database and feature dimension of multi-attribute information of drugs are shown in Table 1. Based on the adverse drug-drug interactions data and multi-attribute data of drugs, 1188258 groups of adverse drug-drug interactions data were collected, including 59377 drug pairs with adverse interactions, N=567 kinds of drugs, and K=258 kinds of adverse interactions, which covering common drugs and adverse interactions. The data collected by this method is reliable. Given drug collection D={d₁, d₂,. . . , d_{N}}, according to adverse interactions between drugs dᵢ and dⱼ, vector r^{ij}∈{0,1}^{K} is constructed to represent an adverse interaction relationship between dᵢ and dⱼ. If the k-th adverse interaction occurs between dᵢ and dⱼ, rₖ^{ij}=1. Otherwise rₖ^{ij}=0.

TABLE 1
Source database and feature dimension
of multi-attribute information of drugs

| | | | Feature |
|---|---|---|---|
| m | Drug attributes | Source database | dimension Lₘ |
| 1 | Molecular structure | DrugBank | 881 |
| 2 | Target | DrugBank | 497 |
| 3 | Pathway | KEGG | 396 |
| 4 | Side effect | SIDER | 3687 |
| 5 | Phenotype | CTD | 2193 |
| 6 | Disease | KEGG | 482 |

The present invention builds a prediction model for adverse drug-drug interactions using molecular structure, targets, pathways, side effects, phenotypes and diseases (Table 1). M represents the number of attributes. In this embodiment, M=6. Matrix X^{m}∈□^{N×L}^{ₘ} is used to represent feature space of the m-th attribute of drugs, and Lₘ is used to represent feature dimension of the m-th attribute of drugs. Take the feature space of molecular structure of drugs as an example (m=2). A relationship between the drug and target is collected in the DrugBank database, and feature space X²∈□^{N×L}^{₂} of target information of drugs is constructed. The feature dimension of the target is L₂=497. Therefore, the target information of the drug dᵢ can be represented by a 497-dimensional binary vector. If the drug dᵢ is related to the j-th target, Xᵢⱼ²=1. Otherwise, Xᵢⱼ²=0. In addition, due to the absence of different attribute information of drugs, that is, if the feature information of the m-th attribute of drug dⱼ has not been recorded in the drug attribute database, X_{j.}^{m}=0^{L}^{ₘ}, where 0^{L}^{ₘ} represents an all-0 vector with a dimension Lₘ and X_{j.}^{m} represents the j-th row of matrix X^{m}. Therefore,

$${X}_{E}^{m} ∈ {▯}^{{N}_{E}^{m} \times {L}_{m}} and {X}_{U}^{m} = {0}^{{N}_{U}^{m} \times {L}_{m}}$$

represent known feature information and absent feature information in the feature space of the m-th attribute of drugs, respectively, and N_{E}^{m} and N_{U}^{m} represent the number of drugs with known feature information and the number of drugs with absent feature information in the feature space of the m-th attribute, respectively.

N_{E}^{m}+N_{U}^{m}=N.

S2: employing multi-attribute information of drugs and constructing the recovery model of multi-attribute absent feature of drugs;
the specific step of constructing the recovery model of multi-attribute absent feature of drugs comprises:
employing multi-attribute information of drugs and constructing a basic model based on the relationship between the common features and unique features of an attribute and its original feature space; and
using KL divergence to measure a distribution difference between unique features of different attributes, and processing the basic model by the distribution difference to obtain the recovery model of multi-attribute absent feature of drugs based on common features and unique features.

The recovery model of multi-attribute absent feature of drugs is built in step S2. The common features and unique features of multi-attribute information of drugs are explored in the recovery model. The common features refer to consistent contribution information for adverse drug-drug interactions prediction in different attributes, and the unique features refer to specific information of different attributes, which is a supplement to adverse interaction prediction. In this step, a basic model based on the relationship between the common features and unique features of an attribute and its original feature space is constructed, and an equality constraint between the feature space X^{m} of the m-th attribute of drugs and its known feature information X_{E}^{m} is introduced to ensure that the known attribute feature information X_{E}^{m} remains unchanged during the recovering process of attribute features, so as to improve the effectiveness of the recovery of attribute features. Therefore, the basic model constructed is a objective function of recovery model of multi-attribute absent feature. The objective function of the recovery model of multi-attribute absent feature of drugs based on common features and unique features may be:

$${min}_{{X}^{m} , P , {Q}^{m} , {U}^{m}} {\sum }_{m = 1}^{M} [ {‖ {X}^{m} - ( P + {Q}^{m} ) {U}^{m} ‖}_{F}^{2} + {α}^{m} {‖ {U}^{m} ‖}_{0} ]$$

$$s . t . {H}_{E}^{m} {X}^{m} = {X}_{E}^{m} , P \geq 0 , {Q}^{m} \geq 0 , {U}^{m} \geq 0 , m = 1 , … , M$$

∥·∥_{F}² represents the Frobenius norm of the matrix; ∥·∥₀ represents the l₀ norm of the matrix, i.e., the number of non-zero elements in the matrix; the matrix P∈□^{N×L} represents the common features of multi-attribute information of drugs; the matrix Q^{m}∈□^{N×L} represents the unique features of the m-th attribute of drugs. L represents dimensions of common features and unique features; U^{m}∈□^{L×L}^{ₘ} represents a reconstruction coefficient matrix of original feature space X^{m}—based on the common features and unique features in the m-th attribute. For the m-th attribute, the number of features of drugs is limited and far less than Lₘ, i.e., the feature dimension of the m-th attribute, so the feature space of attributes of drugs is very sparse. Therefore, the 0-norm constraint of the coefficient matrix U^{m} in formula (1) is introduced to control the sparsity of the reconstruction matrix (P+Q^{m})U^{m} of the original feature space based on common features and unique features, and α^{m} represents a sparsity regularization parameter of the coefficient matrix U^{m}. In the constraint condition,

$${H}_{E}^{m} ∈ {\{ 0 , 1 \}}^{{N}_{E}^{m} \times N}$$

represents a marker matrix of the known feature information X_{E}^{m} of the m-th attribute of drugs, which is obtained by deleting rows corresponding to the index of drugs with absent features from the identity matrix I_{N×N}. H_{E}^{m}X^{m}=X_{E}^{m} means that drugs with known features are extracted from X^{m} and sorted by index to obtain X_{E}^{m}. Constraints P≥0, Q^{m}≥0 and U^{m}≥0 are used to maintain the non-negativity of the matrix.

Formula (1) shows that this step decomposes the feature space of attributes of drugs into common features P and unique features Q^{m} due to the difference in the feature space X^{m} of different drug attributes, m=1, . . . , M. Feature space of all attributes shares the same common features P, and feature space X^{m} of different attribute has there own unique features Q^{m}. Common features and unique features are reconstructed by using a sparse coefficient matrix U^{m}. Owning to the unique features of the feature space X^{m} of different attributes contain the unique information of their attribute space and are not shared with other attribute space, then this step further restricts the specificity of the unique features of different attributes to provide specific inter-attribute complementary information for the prediction of adverse drug-drug interactions based on multi-attribute information. KL (Kullback Leible) divergence is introduced to measure the distribution difference between unique features of different attributes:

$$max {\sum }_{n , m}^{M} [ KL ( {Q}^{m} ‖ {Q}^{n} ) + KL ( {Q}^{n} ‖ {Q}^{m} ) ] \qquad ( 2 )$$

$$KL ( {Q}^{m} ‖ {Q}^{n} ) = {\sum }_{i , j} {Q}_{ij}^{m} log \frac{{Q}_{ij}^{m}}{{Q}_{ij}^{n}}$$

is used to measures the degree of difference between two unique features Q^{m} and Qⁿ, KL(Q^{m}∥Qⁿ)≥0. The smaller the difference between Q^{m} and Qⁿ, the smaller the value of KL divergence. If Q^{m} and Qⁿ are the same, KL(Q^{m}∥Qⁿ)=0. Therefore, the recovery model of multi-attribute absent feature of drugs is obtained by measuring the difference of specificity between the unique matrices of different attributes. The objective function may be:

$${min}_{{X}^{m} , P , {Q}^{m} , {U}^{m}} {\sum }_{m = 1}^{M} [ {‖ {X}^{m} - ( P + {Q}^{m} ) {U}^{m} ‖}_{F}^{2} + {α}^{m} {‖ {U}^{m} ‖}_{0} ] - β {\sum }_{n , m}^{M} [ KL ( {Q}^{m} ‖ {Q}^{n} ) + KL ( {Q}^{n} ‖ {Q}^{m} ) ] \qquad ( 3 )$$

$$s . t . {H}_{E}^{m} {X}^{m} = {X}_{E}^{m} , P \geq 0 , {Q}^{m} \geq 0 , {U}^{m} \geq 0 , m = 1 , … , M$$

∥·∥_{F}² represents the Frobenius norm of the matrix, ∥·∥₀ represents the l₀ norm of the matrix, P represents the common features of multi-attribute information of drugs, Q^{m} represents the unique features of the m-th attribute of drugs, U^{m} represents a reconstruction coefficient matrix of original feature space X^{m} based on the common features and unique features in the m-th attribute, X^{m} represents the feature space of the m-th attribute of drugs, X_{E}^{m} represents the known feature information of the m-th attribute of drugs, KL represents the divergence,

$${H}_{E}^{m} ∈ {\{ 0 , 1 \}}^{{N}_{E}^{m} \times N}$$

represents the marker matrix of X_{E}^{m}, H_{E}^{m}X^{m}=X_{E}^{m} represents the drugs with known features are extracted from X^{m} and sorted by index to obtain X_{E}^{m}. α^{m} represents the sparse regularization parameter of the reconstruction coefficient matrix of the m-th attribute, β represents the regularization parameter of KL divergence between unique features of different attributes.

S3: correcting the recovery model of multi-attribute absent feature of drugs by a cosine similarity regularization term, and solving the corrected recovery model by Lagrange function, alternating direction method of multipliers and nonnegative matrix factorization to obtain the common features and unique features of multi-attribute information of drugs.

The specific method for obtaining the common features and unique features of multi-attribute information of drugs includes:
correcting the recovery model of multi-attribute absent feature of drugs by a cosine similarity regularization term to obtain a corrected model;
solving the corrected model by Lagrange function, alternating direction method of multipliers and nonnegative matrix factorization to obtain the update solutions of recovered feature spaces of different attributes and common features and unique features of multi-attribute information, as well as the reconstruction coefficient matrices of the feature spaces of different attributes; and
iteratively updating variables of the corrected model until it reaches the maximum number of iteration or the difference of the objective function of the model less than a threshold to obtain the common features and unique features of the multi-attribute information of drugs.

In step S2, the feature dimension of the original feature space X^{m} of the m-th attribute is Lₘ, and the dimension of the obtained common feature P and unique feature Q^{m} is L. Therefore, decomposing the feature space X^{m} into common features P and unique features Q^{m} can be regarded as mapping high-dimensional sparse feature space X^{m} to low-dimensional feature space containing common features and unique features of attribute space. For drug dᵢ, X_{i.}^{m} represents the feature information of the m-th attribute of drug dᵢ. The feature representation of dᵢ in low-dimensional space may be constituted by a common feature representation P_{i.} in all attributes and a unique feature representation Q_{i.}^{m} of the m-th attribute of drug dᵢ. Therefore, based on the graph manifold regularization method, the feature representation of drugs in the low-dimensional feature space needs to retain the local geometry structure of the original attribute feature space, that is, in the low-dimensional feature space, feature representation similarity between drugs dᵢ and dⱼ is consistent with that in the original attribute feature space. The feature representation similarity between drugs dᵢ and dⱼ in the feature space of the m-th attribute may be expressed as:

$${S}^{m} ( {d}_{i} , {d}_{j} ) = \frac{〈 {X}_{i \cdot }^{m} , {X}_{j \cdot }^{m} 〉}{{‖ {X}_{i \cdot }^{m} ‖}_{2} {‖ {X}_{j \cdot }^{m} ‖}_{2}} \qquad ( 4 )$$

<X_{i.}^{m}, X_{j.}^{m}> represents the inner product of vectors X_{i.}^{m} and X_{j.}^{m}, and S^{m}(dᵢ, dⱼ) can be regarded as normalized cosine similarity between vectors X_{i.}^{m} and X_{j.}^{m}. In the low-dimensional feature space, (P+Q^{m})_{i.} and (P+Q^{m})_{j.} are a combination representation of common features and unique features of drugs dᵢ and dⱼ respectively, so the regular term of local geometric structure consistency in the attribute feature space of drugs based on cosine similarity can be expressed as:

$${\sum }_{m = 1}^{M} {\sum }_{i , j}^{N} {‖ {( P + {Q}^{m} )}_{i \cdot } - {( P + {Q}^{m} )}_{j \cdot } ‖}_{2}^{2} {S}^{m} ( {d}_{i} , {d}_{j} ) \qquad ( 5 )$$

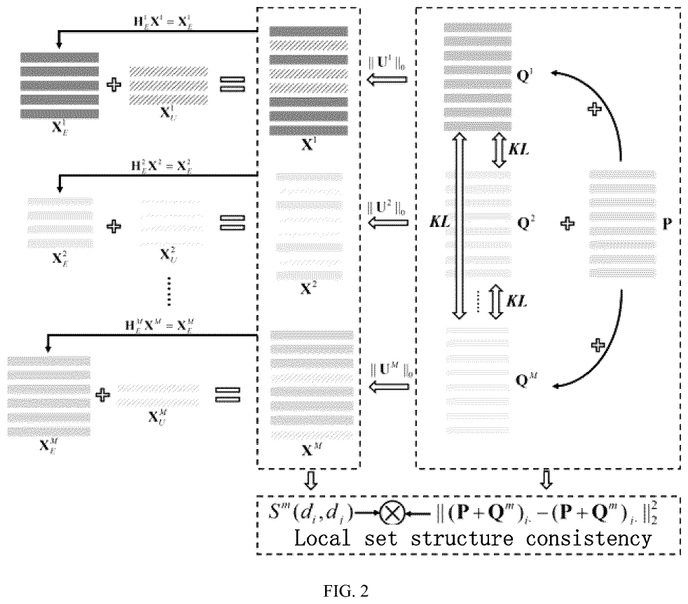

The final model framework of recovery model of multi-attribute absent feature of drugs is shown in FIG. 2, and the final objective function is shown in Formula (6):

$${min}_{{X}^{m} , P , {Q}^{m} , {U}^{m}} {\sum }_{m = 1}^{M} [ {‖ {X}^{m} - ( P + {Q}^{m} ) {U}^{m} ‖}_{F}^{2} + {α}^{m} {‖ {U}^{m} ‖}_{0} + {γ}^{m} {\sum }_{i , j}^{N} {‖ {( P + {Q}^{m} )}_{i \cdot } - {( P + {Q}^{m} )}_{j \cdot } ‖}_{2}^{2} {S}^{m} ( {d}_{i} , {d}_{j} ) ] - β {\sum }_{n , m}^{M} [ KL ( {Q}^{m} ‖ {Q}^{n} ) + KL ( {Q}^{n} ‖ {Q}^{m} ) ] \qquad ( 6 )$$

$$s . t . {H}_{E}^{m} {X}^{m} = {X}_{E}^{m} , P \geq 0 , {Q}^{m} \geq 0 , {U}^{m} \geq 0 , m = 1 , … , M$$

γ^{m} represents a regularization parameter of cosine similarity regularization term of the m-th attribute, and ∥·∥₂² represents the l₂ norm of the vector.

The recovered attribute feature space X^{m} and the common features P and unique features Q^{m} of multi-attribute feature space of drugs, as well as the iterative updating formula of the reconstruction coefficient matrix U^{m} of the attribute feature space are obtained based on augmented Lagrange function, alternating direction method of multipliers and nonnegative matrix factorization optimization method. The maximum number of iterations or the difference threshold of the objective function are set to iteratively update the above model variables, so as to obtain the optimal solution of the model variables and the common features and unique features of multi-attribute information of drugs.

S4: constructing a prediction model based on the common features and unique features of multi-attribute information of drugs and the adverse drug-drug interactions data;

Based on the common features P and unique features Q^{m} of multi-attribute information of drugs obtained in step 3, the prediction model of adverse drug-drug interactions based on multiple attributes was established, and the potential rules between multiple attributes and adverse interactions were revealed by exploring the influence of different attributes on the prediction of adverse drug-drug interactions.

Binary vector r^{ij}∈{0,1}^{K} represents the relationship of adverse drug-drug interactions between drugs dᵢ and dⱼ. Vectors P_{i.} and P_{j.} represent the common features of multiple attributes of dᵢ and dⱼ, respectively, and vectors Q_{i.}^{m} and Q_{j.}^{m} represent the unique features of the m-th attribute of dᵢ and dⱼ, respectively, based on the common features and unique features of the multi-attribute information of drugs optimized in step 3. Since feature space of different attribute of drugs share the same common features and have unique features, adverse interactions between drugs dᵢ and dⱼ can be caused by the common features and unique features. Therefore, the overall objective function of the prediction model for adverse drug-drug interactions based on common features and unique features may be:

$${\sum }_{{\vert {r}^{ij} ‖}_{0} \neq 0} {‖ {r}^{ij} - ( {\overline{r}}^{ij} + {\tilde{r}}^{ij} ) ‖}_{2}^{2} \qquad ( 7 )$$

[symbol]

represents contribution of common features to adverse drug-drug interactions, [symbol] represents contribution of unique features to adverse drug-drug interactions. A tensor Ē∈□^{L×L×K} of common feature-adverse interactions is introduced to estimate the vector [symbol] A tensor element Ēᵢⱼₖ represents the potential relationship between the i-th common feature and the j-th common feature and the k-th adverse interaction. Therefore, the vector [symbol] can be expressed as:

[symbol]

=λ×E̅×₁P_{i.}×₂P_{j.} (8)

The parameter λ represents contribution of the common features P_{i.} and P_{j.} to the adverse interactions between dᵢ and dⱼ, and xₖ represents the product of the k-th order of the tensor and the vector, where k∈{1,2}. On the other hand, the tensor Eₘ is constructed to represent the potential relationship between the unique features of the m-th attribute and adverse interactions since each attribute has unique features. Therefore, vector [symbol] can be contributed by the unique features of each of M attributes:

$${\tilde{r}}^{ij} = {\sum }_{m = 1}^{M} {w}_{m} \times {E}_{m} {\times }_{1} {Q}_{i \cdot }^{m} {\times }_{2} {Q}_{j \cdot }^{m} \qquad ( 9 )$$

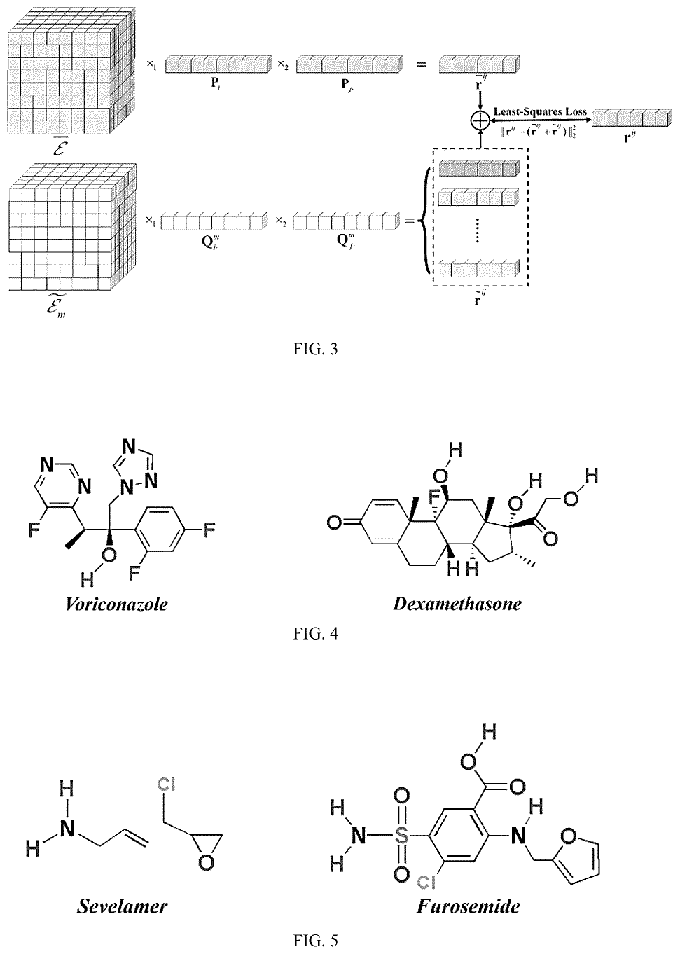

The parameter wₘ represents contribution of the unique features Q_{i.}^{m} and Q_{j.}^{m} of the m-th attribute to the adverse interactions between dᵢ and dⱼ. Therefore, the framework diagram of the prediction model for adverse drug-drug interactions based on common features and unique features is shown in FIG. 3.

Tensors Ē and Eₘ are decomposed into tensor with rank 1 to estimate the adverse interactions r^{ij} between drugs dᵢ and dⱼ based on the high order tensor-low rank CP decomposition method. The implicit parameters of the model are optimized iteratively by random gradient descent method to update the parameters of the model.

S5: obtaining the multi-attribute information of two drugs, calculating common features and unique features of their multi-attribute information as the inputs of the prediction model to predict their adverse drug-drug interactions.

Given multi-attribute information X_{a.}^{m} and X_{b.}^{m} of any two drugs dₐ and d_{b} to predict the adverse interactions between drugs dₐ and d_{b}. The absent attribute features of drugs dₐ and d_{b} are recovered to obtain their common features and unique features based on the recovery model frame of multi-attribute feature of drugs. According to the prediction model of adverse drug-drug interactions based on common features and unique features, the prediction of adverse interactions between dₐ and d_{b} can be expressed as:

$${\overline{r}}^{ab} + {\tilde{r}}^{ab} = λ \times \overline{E} {\times }_{1} {P}_{a \cdot } {\times }_{2} {P}_{b \cdot } + {\sum }_{m = 1}^{M} {w}_{m} \times {E}_{m} {\times }_{1} {Q}_{a \cdot }^{m} {\times }_{2} {Q}_{b \cdot }^{m} \qquad ( 10 )$$

In the present embodiment, the adverse drug-drug interactions can be predicted by the proposed adverse drug-drug interaction prediction method by recovering the multi-attribute information of drugs, and some prediction results are supported by relevant literature. The prediction results can provide data support for the study of adverse drug-drug interactions and the study of new drug safety based on biological experimental methods. FIG. 4 and FIG. 5 show some prediction results of adverse drug-drug interactions. The FIG. 4 shows that the combination of voriconazole (triazole antifungal agent for preventing aspergillosis and candida infection) and dexamethasone (synthetic adrenal corticosteroid for treating rheumatoid arthritis, brain edema and acute pulmonary edema) will cause adverse interactions such as sepsis, visual impairment and osteoporosis. The cause of adverse interactions is that the side effects of the two drugs are similar, and some of the substructures act on the same target, pathway and disease. The FIG. 5 shows that the combination of sevelamer (unabsorbable polyamine for preventing hyperphosphatemia) and furanilic acid (sulfamethylaminobenzoic acid derivatives for treating congestive heart failure) will cause adverse interactions such as cardiac arrest, bradycardia and non dynamic intestinal obstruction.

This embodiment discloses the method for predicting adverse interactions by recovering multi-attribute information of drugs, which recovers the absent features of drugs by constructing the recovery model of multi-attribute absent feature of drugs, and then predicts adverse interactions among drugs with the recovered attribute features. This not only improves the accuracy of the prediction of the adverse drug-drug interactions, but also promotes the experimental study of the adverse drug-drug interactions, and ensures the safety of medication.

### Embodiment 2

This embodiment discloses a system for predicting adverse drug-drug interactions by recovering multi-attribute absent feature. This embodiment aims to realize the prediction method in Embodiment 1, including the data collecting module, the recovery model construction module, the analysis module, the prediction model construction module and the prediction module; wherein, the data collecting module is configured to collecting adverse drug-drug interactions data and multi-attribute data of drugs; the recovery model construction module is configured to construct a recovery model of multi-attribute absent feature of drugs based on common features and unique features of multi-attribute data of drugs; the analysis module is configured to correct the recovery model by a cosine similarity regularization term, and solve the corrected recovery model by Lagrange function, alternating direction method of multipliers and nonnegative matrix factorization to obtain the common features and unique features of the multi-attribute information of drugs; the prediction model construction module is configured to construct a prediction model based on the common features and unique features of multi-attribute information of drugs and the adverse drug-drug interactions data; and the prediction module is configured to obtain the multi-attribute information of two drugs, calculate common features and unique features of their multi-attribute information as the inputs of the prediction model to predict their adverse drug-drug interactions.

### Embodiment 3

This embodiment discloses the computer storage medium storing the computing program, where the method described in Embodiment 1 is implemented when the computer program is executed by the processor.

The person skilled in the art should understand that the embodiments of this application may be provided as a method, a system, or a computer program product. Therefore, this application may use a form of hardware only embodiments, software only embodiments, or embodiments with a combination of software and hardware. Moreover, this application may use a form of a computer program product that is implemented on one or more computer-usable storage media (including but not limited to a magnetic disk memory, a CD-ROM, an optical memory, and the like) that include computer usable program code.

The present invention is described with reference to the flowcharts and/or block diagrams of the method, the device (system), and the computer program product according to the embodiments of this application. It should be understood that computer program issuing instructions may be used to implement each process and/or each block in the flowcharts and/or the block diagrams and a combination of a process and/or a block in the flowcharts and/or the block diagrams. These computer program issuing instructions may be provided for a general-purpose computer, a dedicated computer, an embedded processor, or a processor of any other programmable data processing device to generate a machine, so that the issuing instructions executed by a computer or a processor of any other programmable data processing device generate an apparatus for implementing a specific function in one or more processes in the flowcharts and/or in one or more blocks in the block diagrams.

These computer program issuing instructions may be stored in a computer readable memory that can instruct the computer or any other programmable data processing device to work in a specific manner, so that the issuing instructions stored in the computer readable memory generate an artifact that includes an issuing instruction apparatus. The issuing instruction apparatus implements a specific function in one or more processes in the flowcharts and/or in one or more blocks in the block diagrams.

These computer program issuing instructions may be loaded onto a computer or another programmable data processing device, so that a series of operations and steps are performed on the computer or the another programmable device, to generate computer-implemented processing. Therefore, the issuing instructions executed on the computer or the another programmable device provide steps for implementing a specific function in one or more processes in the flowcharts and/or in one or more blocks in the block diagrams.

What is claimed is:

1. A method for predicting adverse drug-drug interactions by recovering the multi-attribute information of drugs, wherein the method comprises:
collecting adverse drug-drug interactions data and multi-attribute data of drugs;
constructing a recovery model of multi-attribute absent feature of drugs based on common features and unique features of multi-attribute information of drugs;
correcting the recovery model of multi-attribute absent feature of drugs by a cosine similarity regularization term, and solving the corrected recovery model by Lagrange function, alternating direction method of multipliers and nonnegative matrix factorization to obtain the common features and unique features of multi-attribute information of drugs;
constructing a prediction model based on the common features and unique features of multi-attribute information of drugs and the adverse drug-drug interactions data; and
obtaining the multi-attribute information of two drugs, calculating common features and unique features of their multi-attribute information as the inputs of the prediction model to predict their adverse drug-drug interactions.

2. The method for predicting adverse drug-drug interactions by recovering multi-attribute information of drugs according to claim 1, wherein constructing the recovery model of multi-attribute absent feature of drugs comprises:
employing multi-attribute information of drugs and constructing a basic model based on the relationship between the common features and unique features of an attribute and its original feature space; and
using KL divergence to measure a distribution difference between unique features of different attributes, and processing the basic model by the distribution difference to obtain the recovery model of multi-attribute absent feature of drugs based on common features and unique features.

3. The method for predicting adverse drug-drug interactions by recovering multi-attribute information of drugs according to claim 1, wherein a specific method for obtaining the common features and unique features of the multi-attribute information of drugs comprises:
correcting the recovery model of multi-attribute absent feature of drugs by a cosine similarity regularization term to obtain a corrected model;
solving the corrected model by Lagrange function, alternating direction method of multipliers and nonnegative matrix factorization to obtain the update solutions of recovered feature spaces of different attributes and common features and unique features of multi-attribute information, as well as the reconstruction coefficient matrices of the feature spaces of different attributes; and
iteratively updating variables of the corrected model until it reaches the maximum number of iteration or the difference of the objective function of the model less than a threshold to obtain the common features and unique features of the multi-attribute information of drugs.

4. The method for predicting adverse drug-drug interactions by recovering multi-attribute information of drugs according to claim 3, wherein the multi-attribute information of drugs comprises molecular structure, target, pathway, side effect, phenotype, and disease data.

5. The method for predicting adverse drug-drug interactions by recovering multi-attribute information of drugs according to claim 3, wherein a specific expression of the recovery model of multi-attribute absent feature of drugs based on common features and unique features is:
$${min}_{{X}^{m} , P , {Q}^{m} , {U}^{m}} {\sum }_{m = 1}^{M} [ {‖ {X}^{m} - ( P + {Q}^{m} ) {U}^{m} ‖}_{F}^{2} + {α}^{m} {‖ {U}^{m} ‖}_{0} ] - β {\sum }_{n , m}^{M} [ KL ( {Q}^{m} ‖ {Q}^{n} ) + KL ( {Q}^{n} ‖ {Q}^{m} ) ]$$
$$s . t . {H}_{E}^{m} {X}^{m} = {X}_{E}^{m} , P \geq 0 , {Q}^{m} \geq 0 , {U}^{m} \geq 0 , m = 1 , … , M$$
∥·∥_{F}² represents the Frobenius norm of the matrix, ∥·∥₀ represents the l₀ norm of the matrix, P represents the common features of multi-attribute information of drugs, Q^{m} represents the unique features of the m-th attribute of drugs, U^{m} represents a reconstruction coefficient matrix of original feature space X^{m} based on the common features and unique features in the m-th attribute, X^{m} represents original feature space of the m-th attribute of drugs, X_{E}^{m} represents the known feature information of the m-th attribute of drugs, KL represents divergence,
$${H}_{E}^{m} ∈ {\{ 0 , 1 \}}^{{N}_{E}^{m} \times N}$$
represents a marker matrix of X_{E}^{m}, H_{E}^{m}X^{m}=X_{E}^{m} represents that drugs with known features are extracted from X^{m} and sorted by index to obtain X_{E}^{m}. α^{m} represents a sparse regularization parameter of the reconstruction coefficient matrix of the m-th attribute, β represents a regularization parameter of KL divergence between unique features of different attributes.

6. The method for predicting adverse drug-drug interactions by recovering multi-attribute information of drugs according to claim 5, wherein a specific expression of the corrected model is:
$${min}_{{X}^{m} , P , {Q}^{m} , {U}^{m}} {\sum }_{m = 1}^{M} [ {‖ {X}^{m} - ( P + {Q}^{m} ) {U}^{m} ‖}_{F}^{2} + {α}^{m} {‖ {U}^{m} ‖}_{0} + {γ}^{m} {\sum }_{i , j}^{N} {‖ {( P + {Q}^{m} )}_{i \cdot } - {( P + {Q}^{m} )}_{j \cdot } ‖}_{2}^{2} {S}^{m} ( {d}_{i} , {d}_{j} ) ] - β {\sum }_{n , m}^{M} [ KL ( {Q}^{m} ‖ {Q}^{n} ) + KL ( {Q}^{n} ‖ {Q}^{m} ) ]$$
$$s . t . {H}_{E}^{m} {X}^{m} = {X}_{E}^{m} , P \geq 0 , {Q}^{m} \geq 0 , {U}^{m} \geq 0 , m = 1 , … , M$$
S^{m}(dᵢ,dⱼ) can be regarded as standardized cosine similarity between vectors X_{i.}^{m} and X_{j.}^{m}, (P+Q^{m})_{i.} and (P+Q^{m})_{j.} are a combined representation of common features and unique features of drugs dᵢ and dⱼ, respectively, γ^{m} represents a regularization parameter of cosine similarity regularization term of the m-th attribute, and ∥·∥₂² represents the l₂ norm of a vector.

7. The method for predicting adverse drug-drug interactions by recovering multi-attribute information of drugs according to claim 1, wherein a specific expression of the prediction model is:
$${\sum }_{{\vert {r}^{ij} ‖}_{0} \neq 0} {‖ {r}^{ij} - ( {\overline{r}}^{ij} + {\tilde{r}}^{ij} ) ‖}_{2}^{2}$$ [symbol]
represents contribution of common features to adverse drug-drug interactions, [symbol] represents contribution of unique features to adverse drug-drug interactions, and r^{ij} represents a relationship of adverse drug-drug interactions.

8. The method for predicting adverse drug-drug interactions by recovering multi-attribute information of drugs according to claim 7, wherein a specific expression of [symbol] is: [symbol]
=λ×E̅×₁P_{i.}×₂P_{j.}
a specific expression of [symbol] is:
$${\tilde{r}}^{ij} = {\sum }_{m = 1}^{M} {w}_{m} \times {E}_{m} {\times }_{1} {Q}_{i \cdot }^{m} {\times }_{2} {Q}_{j \cdot }^{m}$$
P_{i.} represents common features of multi-attribute of drug dᵢ, P_{j.} represents common features of multi-attribute of drug dⱼ, Q_{i.}^{m} represents unique features of the m-th attribute of drug dᵢ, Q_{j.}^{m} represents unique features of the m-th attribute of drug dⱼ, wₘ represents contribution of unique features Q_{i.}^{m} and Q_{j.}^{m} of the m-th attribute to adverse interactions between dᵢ and dⱼ, λ represents contribution of common features P_{i.} and P_{j.} to the adverse interactions between dᵢ and dⱼ, Ē represents tensor of the common features—adverse interactions that indicates a potential relationship between the common features and adverse interactions, Eₘ represents a potential relationship between the unique features of the m-th attribute and adverse interactions, and xₖ represents the product of the k-th order of the tensor and the vector, where k∈{1,2}.

9. The method for predicting adverse drug-drug interactions by recovering multi-attribute information of drugs according to claim 2, wherein a specific method for obtaining the common features and unique features of the multi-attribute information of drugs comprises:
correcting the recovery model of multi-attribute absent feature of drugs by a cosine similarity regularization term to obtain a corrected model;
solving the corrected model by using Lagrange function, alternating direction method of multipliers and nonnegative matrix factorization to obtain a recovered feature space of attributes and common features and unique features of feature space of multi-attribute information, as well as an iterative updating formula of a reconstruction coefficient matrix of the feature space; and
iteratively updating variables of the corrected model until it reaches the maximum number of iteration or the difference of the objective function of the model less than a threshold to obtain the common features and unique features of the multi-attribute information of drugs.

10. The method for predicting adverse drug-drug interactions by recovering multi-attribute information of drugs according to claim 9, wherein the multi-attribute data of drugs comprises molecular structure, target, pathway, side effect, phenotype, and disease data.

11. The method for predicting adverse drug-drug interactions by recovering multi-attribute information of drugs according to claim 9, wherein a specific expression of the recovery model of multi-attribute absent feature of drugs based on common features and unique features is:
$${min}_{{X}^{m} , P , {Q}^{m} , {U}^{m}} {\sum }_{m = 1}^{M} [ {‖ {X}^{m} - ( P + {Q}^{m} ) {U}^{m} ‖}_{F}^{2} + {α}^{m} {‖ {U}^{m} ‖}_{0} ] - β {\sum }_{n , m}^{M} [ KL ( {Q}^{m} ‖ {Q}^{n} ) + KL ( {Q}^{n} ‖ {Q}^{m} ) ]$$
$$s . t . {H}_{E}^{m} {X}^{m} = {X}_{E}^{m} , P \geq 0 , {Q}^{m} \geq 0 , {U}^{m} \geq 0 , m = 1 , … , M$$
∥·∥_{F}² represents the Frobenius norm of the matrix, ∥·∥₀ represents the l₀ norm of the matrix, P represents the common features of multi-attribute information of drugs, Q^{m} represents the unique features of the m-th attribute of drugs, U^{m} represents a reconstruction coefficient matrix of original feature space X^{m} based on the common features and unique features in the m-th attribute, X^{m} represents original feature space of the m-th attribute of drugs, X_{E}^{m} represents the known feature information of the m-th attribute of drugs, KL represents divergence,
$${H}_{E}^{m} ∈ {\{ 0 , 1 \}}^{{N}_{E}^{m} \times N}$$
represents a marker matrix of X_{E}^{m}, H_{E}^{m}X^{m=X}_{E}^{m} represents that drugs with known features are extracted from X^{m} and sorted by index to obtain X_{E}^{m}. α^{m} represents a sparse regularization parameter of the reconstruction coefficient matrix of the m-th attribute, β represents a regularization parameter of KL divergence between unique features of different attributes.

12. The method for predicting adverse drug-drug interactions by recovering multi-attribute information of drugs according to claim 11, wherein a specific expression of the corrected model is:
$${min}_{{X}^{m} , P , {Q}^{m} , {U}^{m}} {\sum }_{m = 1}^{M} [ {‖ {X}^{m} - ( P + {Q}^{m} ) {U}^{m} ‖}_{F}^{2} + {α}^{m} {‖ {U}^{m} ‖}_{0} + {γ}^{m} {\sum }_{i , j}^{N} {‖ {( P + {Q}^{m} )}_{i \cdot } - {( P + {Q}^{m} )}_{j \cdot } ‖}_{2}^{2} {S}^{m} ( {d}_{i} , {d}_{j} ) ] - β {\sum }_{n , m}^{M} [ KL ( {Q}^{m} ‖ {Q}^{n} ) + KL ( {Q}^{n} ‖ {Q}^{m} ) ]$$
$$s . t . {H}_{E}^{m} {X}^{m} = {X}_{E}^{m} , P \geq 0 , {Q}^{m} \geq 0 , {U}^{m} \geq 0 , m = 1 , … , M$$
S^{m}(dᵢ,dⱼ) can be regarded as standardized cosine similarity between vectors X_{i.}^{m} and X_{j.}^{m}, (P+Q^{m})_{i.} and (P+Q^{m})_{j.} are a combined representation of common features and unique features of drugs dᵢ and dⱼ, respectively, γ^{m} represents a regularization parameter of cosine similarity regularization term of the m-th attribute, and ∥·∥₂² represents the l₂ norm of a vector.

13. A system for predicting adverse drug-drug interactions by recovering the multi-attribute information of drugs, comprising a data collecting module, a recovery model construction module, an analysis module, a prediction model construction module and a prediction module; wherein,
the data collecting module is configured to collecting adverse drug-drug interactions data and multi-attribute data of drugs;
the recovery model construction module is configured to construct a recovery model of multi-attribute absent feature of drugs based on common features and unique features of multi-attribute data of drugs;
the analysis module is configured to correct the recovery model by a cosine similarity regularization term, and solve the corrected recovery model by Lagrange function, alternating direction method of multipliers and nonnegative matrix factorization to obtain the common features and unique features of the multi-attribute information of drugs;
the prediction model construction module is configured to construct a prediction model based on the common features and unique features of multi-attribute information of drugs and the adverse drug-drug interactions data; and
the prediction module is configured to obtain the multi-attribute information of two drugs, calculate common features and unique features of their multi-attribute information as the inputs of the prediction model to predict their adverse drug-drug interactions.

14. A computer storage medium storing a computing program, wherein the method according to claim 1 is implemented when the computer program is executed by a processor.
